(19) United States
(12) Patent Application Publication
(10) Pub. No.: US 2015/0253214 A1
(43) Pub. Date: Sep. 10, 2015

(54) TEST AND/OR BURN-IN OF LAB-ON-A-CHIP DEVICES

(71) Applicant: Canon U.S. Life Sciences, Inc., Rockville, MD (US)

(72) Inventors: Kenton C. Hasson, Germantown, MD (US); Johnathan S. Coursey, Rockville, MD (US); Gregory H. Owen, Clarksburg, MD (US)

(73) Assignee: Canon U.S. Life Sciences, Inc., Rockville, MD (US)

(21) Appl. No.: 14/640,719

(22) Filed: Mar. 6, 2015

Related U.S. Application Data
(60) Provisional application No. 61/948,865, filed on Mar. 6, 2014.

(51) Int. Cl. G01M 3/02; C12Q 1/68

(52) U.S. Cl. CPC G01M 3/02; C12Q 1/686

(57) ABSTRACT

Systems and methods for testing a fluidic device comprising fluidic features are disclosed. In some embodiments, the systems and methods may perform testing and burn-in of one or more fluidic features of the fluidic device. In some embodiments, the systems and methods may subject one or more of the fluidic features to a differential pressure, measure a pressure response of one or more of the fluidic features to the differential pressure, and detecting whether an abnormality is present in the pressure response. In some embodiments, the systems and methods may perform proof testing one or more fluidic features. The proof testing may include subjecting a fluidic feature to a proof pressure and monitoring the pressure of the fluidic feature for a period of time. A change in pressure at one or more of the waste and vent wells may be indicative of a leak in the fluidic feature.

### CROSS REFERENCE TO RELATED APPLICATION

This application claims the benefit of priority to U.S. Provisional Application Ser. No. 61/948,865, filed on Mar. 6, 2014, which is incorporated herein by reference in its entirety.

### BACKGROUND

1. Field of Invention

The present invention relates to testing and/or burn-in of lab-on-a-chip (LOC) devices or components thereof. More specifically, embodiments of the present invention relate to testing and/or burn-in of LOC devices (or components thereof) having one or more fluidic features.

2. Discussion of the Background

Research and production of lab-on-a-chip (LOC) devices have grown out of the microfabrication and microelectronics industry. LOC devices are one type of micro-electro-mechanical systems (MEMS). Most MEMS are in the research stage, and little attention has been given to scale-up and production issues. One issue with scale-up is testing and validation of devices produced for end-users. While MEMS testing may involve some aspects similar to integrated circuit (IC) chip testing in the semiconductors industry, MEMS devices present further challenges because mechanical, chemical and/or optical parameters may be tested in addition to electrical properties, and detection of failure modes not present in pure electrical systems can be important. See, e.g., Description of the 3rd Annual Conference on MEMS Testing and Reliability held on Oct. 20, 2011 at http://www.memsjournal.com/mtr2011.html; Tai-Ran Hsu, Introduction to Reliability in MEMS Packaging (presented at International Symposium for Testing & Failure Analysis, San Jose, Calif. (Nov. 5, 2007)) (available at www.engr.sjsu.edu/trhsu/ISTFA%20paper%2007.pdf); Chapter 11 Assembly, Packaging, and Testing (APT) of Microsystems (available at www.engr.sjsu.edu/trhsu/ME189_Chapter%2011.pdf); P. Galambos and G. Benavides, Electrical and Fluidic Packaging of Surface Micromachined Electro-Microfluidic Devices, Microfluidic Devices and Systems III, Proceedings of SPIE—The International Society of Optical Engineering, vol. 4177, 2000, pp. 200-207; Ahn, et al., Disposable Smart Lab-On-A-Chip For Point-Of-Care Clinical Diagnostics, Proceedings of the IEEE, Special Issue on Biomedical Applications for MEMS and Microfluidics, 2004, p. 154-173; U.S. Patent Application Publication No. 2007/0105339; U.S. Pat. No. 4,549,248. See also http://www.rheonix.com/technology/rheonix-card-consumable.php; http://www.alineinc.com/.

There is thus a need in the art for improved systems and methods for testing and/or burn-in of LOC devices or components thereof.

### SUMMARY

One aspect of the invention may provide a method of testing a fluidic device including fluidic features. The method may include subjecting one or more of the fluidic features to a differential pressure. The method may include measuring a pressure response of one or more of the fluidic features to the differential pressure. The method may include detecting whether an abnormality is present in the pressure response.

In some embodiments, one or more of the fluidic features may include a channel. In some embodiments, one or more of the fluidic features may include a sample well. In some embodiments, the fluidic device may be a sub-component of a lab-on-a-chip device.

In some embodiments, measuring the pressure response may include measuring the pressure response of one or more fluidic features that were not subjected to the differential pressure. In some embodiments, the differential pressure may be positive. In some embodiments, the differential pressure may be negative.

In some embodiments, the method may include testing one or more electrical features of the fluidic device, and the testing may be performed at the same time as or serially with one or more of subjecting the one or more of the fluidic features to the differential pressure. In some embodiments, the method may include measuring the pressure response and detecting whether the abnormality is present. In some embodiments, the one or more electrical features may include a resistor.

In some embodiments, the method may include subjecting the fluidic device to a thermal profile. In some embodiments, subjecting the fluidic device to the thermal profile may include powering one or more features included in or on the fluidic device. In some embodiments, subjecting the fluidic device to the thermal profile may include using an environmental chamber or heater that is external to the fluidic device. In some embodiments, the thermal profile may include a temperature ramp. In some embodiments, the thermal profile may include one or more temperature steps or PCR temperature cycles.

In some embodiments, the method may include subjecting the fluidic device to a humidity and/or pressure profile. In some embodiments, subjecting the one or more fluidic features to the differential pressure may include applying the differential pressure to two or more fluidic features at the same time. In some embodiments, the method may include introducing a liquid into at least one fluidic feature.

In some embodiments, the method may include passing a current through one or more electrical features of the fluidic device. In some embodiments, the electrical features may include one or more of a heater, sensor, resistor, capacitor, controller, counter, timer, memory, processor, actuator, and valve. In some embodiments, passing the current through the one or more electrical features may include running a burn-in program. In some embodiments, the burn-in program may simulate normal fluidic device usage. In some embodiments, the burn-in program may include running the fluidic device at a temperature higher than a standard operating temperature for the device.

Another aspect of the invention may provide a method for testing a channel in a fluidic device for leakages. The method may include opening a valve in communication with the channel. The channel may be in communication with one or more wells. The method may include subjecting the channel to a proof pressure. The method may include closing the valve. The method may include monitoring pressure at one or more of the wells. A change in pressure at one or more of the wells may be indicative of a leak in the channel.

In some embodiments, the proof pressure may be a negative proof pressure, and an increase in pressure at one or more of the wells may be indicative of a leak in the channel. In some embodiments, the proof pressure may be a positive proof pressure, and a decrease in pressure at one or more of the wells may be indicative of a leak in the channel. In some embodiments, the channel may be in communication with a waste well and a vent well, and monitoring the pressure at one or more of the wells may include monitoring pressure at one or more of the waste and vent wells.

Still another aspect of the invention may provide a system for testing a fluidic device comprising fluidic features. The system may include: one or more valves or accumulators; one or more pressure monitors; a device interface module; and a pressure controller. The device interface module may be configured to hold the fluidic device, connect the one or more valves or accumulators to one or more of the fluidic features, and connect the one or more pressure monitors to one or more of the fluidic features. The pressure controller may be configured to control the one or more valves or accumulators to subject one or more of the fluidic features to a differential pressure and control the one or more pressure monitors to measure a pressure response of one or more of the fluidic features.

In some embodiments, one or more of the fluidic features may include a channel. In some embodiments, one or more of the fluidic features may include a sample well. In some embodiments, the fluidic device is a sub-component of a lab-on-a-chip device, and the device interface module is configured to hold the lab-on-a-chip device. In some embodiments, the differential pressure may be positive. In some embodiments, the differential pressure may be negative.

In some embodiments, the system may include a system controller configured to detect whether an abnormality is present in the pressure response. In some embodiments, the system controller may be configured to control the pressure controller. In some embodiments, the system may include a storage medium, wherein the system controller is configured store test results in the storage medium. In some embodiments, the system may include a graphical user interface, wherein the system controller is configured to present test results to a user using the graphical user interface.

In some embodiments, the system may include a circuit tester configured to test one or more electrical features of the fluidic device. In some embodiments, the system controller may be configured to control the circuit tester to test the one or more electrical features at the same time as or serially with subjecting the one or more of the fluidic features to the differential pressure or measuring the pressure response. In some embodiments, the one or more electrical features may include a resistor. In some embodiments, the system controller may be configured to control the circuit tester to subject the fluidic device to a thermal profile. In some embodiments, subjecting the fluidic device to the thermal profile may include powering one or more features included in or on the fluidic device. In some embodiments, the thermal profile may include a temperature ramp. In some embodiments, the thermal profile may include one or more temperature steps or PCR temperature cycles. In some embodiments, the circuit tester may be configured to pass a current through one or more electrical features of the fluidic device. In some embodiments, the electrical features may include one or more of a heater, sensor, resistor, capacitor, controller, counter, timer, memory, processor, actuator, and valve. In some embodiments, the circuit tester the system controller may be configured to control the electrical tester to burn-in the fluidic device, and the burn-in comprises passing a current through the one or more electrical features of the fluidic device.

In some embodiments, the system may include an environmental chamber or heater that is external to the fluidic device, and the system controller may be configured to subject the fluidic device to a thermal profile by using the environmental chamber or the external heater. In some embodiments, the system may include an environmental controller configured to control the environmental conditions under which testing is performed. In some embodiments, the system controller may be configured to control the environmental controller to subject the fluidic device to a humidity and/or pressure profile.

In some embodiments, subjecting the one or more fluidic features to the differential pressure may include applying the differential pressure to two or more fluidic features at the same time. In some embodiments, the pressure response may be of one or more fluidic features that were not subjected to the differential pressure.

Another aspect of the invention may provide a system for testing a channel in a fluidic device for leakages. The system may include a valve, one or more pressure monitors, a device interface module, a pressure controller, and a system controller. The device interface module may be configured to hold the fluidic device, connect the valve to the channel of the fluidic device, and connect the one or more pressure monitors to one or more wells in communication with the channel. The pressure controller may be configured to open and close the valve, to subject the channel to a proof pressure, and to control the one or more pressure monitors to measure a pressure at one or more of the wells. The system controller may be configured to (i) control the pressure controller to open the valve, subject to the channel to the proof pressure, close the valve, and control the one or more pressure monitors to measure a pressure at one or more of the wells, and (ii) determine whether the measured pressure at one or more of the wells changes. A change in pressure at one or more of the wells may be indicative of a leak in the channel.

The above and other embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements. Additionally, the left-most digit(s) of the reference number identifies the drawing in which the reference number first appears.

FIG. 1 depicts a perspective view of a top, rear, and left side of a lab-on-a-chip (LOC) device embodying aspects of the present invention.

FIG. 2 depicts an exploded, perspective view of a top, front, and left side of an LOC device embodying aspects of the present invention.

FIG. 3 depicts a top view of a fluidic device of an LOC device embodying aspects of the present invention.

FIG. 4 depicts a top view of a reaction chip of a fluidic device of an LOC device embodying aspects of the present invention.

FIG. 5 depicts a schematic diagram illustrating an LOC test and/or burn-in system embodying aspects of the present invention.

FIG. 6 depicts a perspective view of a top, rear, and left side of a closed LOC test and/or burn-in system embodying aspects of the present invention.

FIG. 7 depicts a perspective view of a top and left side of an open LOC test and/or burn-in system embodying aspects of the present invention.

FIG. 8 depicts a perspective view of a portion of a top and left side an LOC test and/or burn-in system embodying aspects of the present invention.

FIG. 9 is a schematic diagram illustrating an LOC test and/or burn-in system configured to test an LOC device 100 according to some embodiments of the invention.

FIGS. 10 and 11 are flowcharts illustrating processes for testing one or more fluidic features of an LOC device or component thereof embodying some aspects of the present invention.

FIGS. 12 and 13 are flowcharts illustrating processes for individually testing one or more fluidic features of an LOC device or component thereof embodying some aspects of the present invention.

FIGS. 14 and 15 are flowcharts illustrating processes for testing one or more sets of fluidic features of an LOC device or component thereof embodying some aspects of the present invention.

FIG. 16 is a flowchart illustrating a process for issuing a test report embodying some aspects of the present invention.

FIGS. 17A-17H illustrate device fixture adaptors that may be used to test different LOC device components embodying some aspects of the present invention.

FIG. 18 is a screenshot illustrating test results that may be presented by an LOC test and/or burn-in system embodying aspects of the present invention.

FIG. 19 is a flowchart illustrating a process for proof testing one or more fluidic features of an LOC device or component thereof embodying some aspects of the present invention.

FIG. 20 is a graph illustrating simulated pressure data from a positive proof pressure test embodying some aspects of the present invention.

FIG. 21 is a table illustrating an example of proof testing of more than one fluidic feature at a time embodying some aspects of the present invention.

### DETAILED DESCRIPTION OF PREFERRED EMBODIMENTS

Scale-up of lab-on-a-chip (LOC) devices and/or bringing these devices to market may involve rigorous testing and validation of each LOC device before it is released to the end user. Testing of the LOC devices or components thereof can avoid failures that, for example, waste the end user's time, money, and/or precious samples. Burn-in is the process of using a device or components thereof prior to being placed in service. Burn-in may involve running a device continuously for a period of time. Burn-in of a device or component thereof may include operating the device in a manner is similar to the way the device or component will be used in service by an end-user. For example, burn-in of a television may involve powering up the television and running through various images for a specified period of time. Some aspects of the present invention may relate to devices and methods for testing and/or burn-in of an LOC device.

LOC devices may include one or more fluidic features (e.g., sample wells, reservoirs, reaction chambers, channels, and channel networks) in addition to other non-fluidic functions (e.g., optical detection, thermal control, electrical sensors and circuitry, memory, etc.). Some embodiments of the present invention may relate to validation of the LOC device may include evaluating one or more of the fluidic features for integrity and reliability. Some embodiments may relate to a testing and evaluation process that can validate fluidic features of LOC devices (e.g., before the devices are released to the end-user).

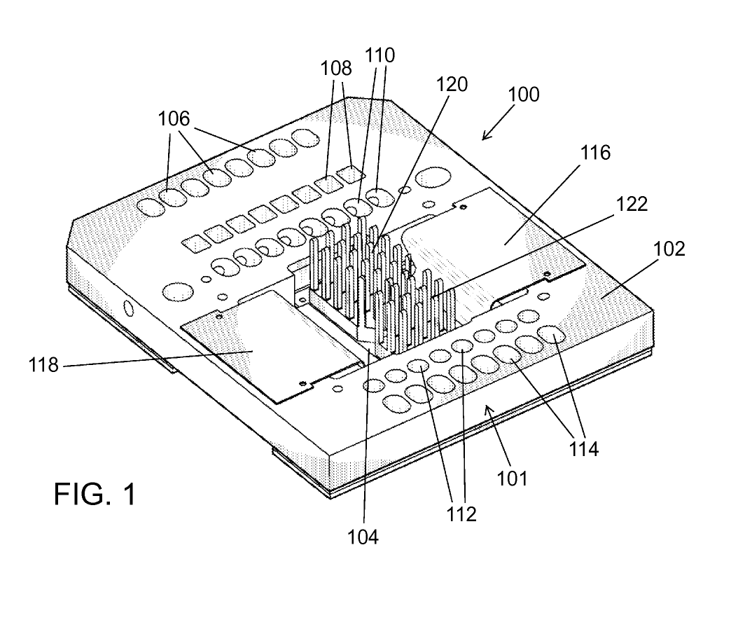

FIGS. 1-4 illustrate an example of an LOC device 100 that may be tested and/or validated using systems and methods in accordance with the present invention. However, systems and methods in accordance with the present invention may be useful with any device that contains at least one fluidic feature. In some non-limiting embodiments, the device may include one or more non-fluidic features, and systems and methods in accordance with the present invention may additionally test or validate one or more of the non-fluidic features.

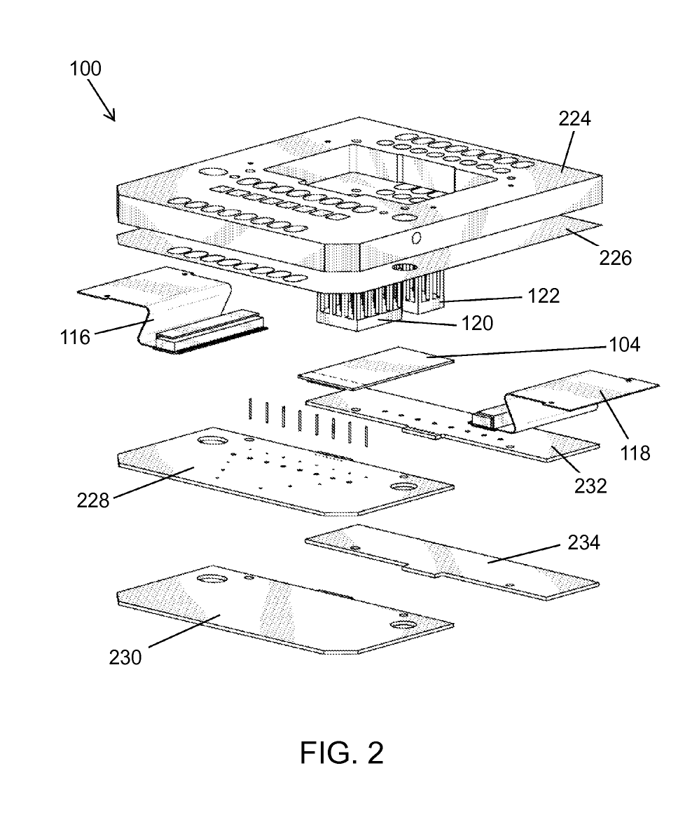

FIG. 1 illustrates a perspective view of the assembled LOC device 100. FIG. 2 illustrates an exploded view of the LOC device 100. In some embodiments, the LOC device 100 may include a fluidic device 101, one or more flexible circuits 116 and 118, and/or one or more heat sinks 120 and 122.

The fluidic device 101 may include one or more fluidic features. A fluidic feature may be, for example and without limitation, a sample well, a buried reservoir, a surface reservoir, a reaction chamber, a channel (e.g., a microchannel), or a channel network (e.g., a microchannel network). In some non-limiting embodiments, the fluidic device may include an interface chip 102 and a reaction chip 104. The interface chip 102 may provide one or more fluids to and from the reaction chip 104. The interface chip 102 may include one or more wells or reservoirs 106 and 114, one or more vent wells or outlets 108, one or more sipper wells or inlets 110, and one or more waste wells or outlets 112. In some non-limiting embodiments, the wells or reservoirs 106 may be sample wells or reservoirs, and the wells or reservoirs 114 may be blanking or spacer fluid wells or reservoirs. In one non-limiting embodiment, the interface chip 102 may include eight sample wells 114, eight vent wells 108, eight inlets 110, eight outlets 112, and eight blanking wells 114. However, this is not required, and some alternative embodiments may include a different number of wells, inlets, and outlets. In some embodiments, the reaction chip 104 may be configured to carry out reaction chemistry, such as, for example and without limitation, one or more of polymerase chain reaction (PCR) thermal cycling for PCR amplification and/or thermal ramping for melting curve analysis.

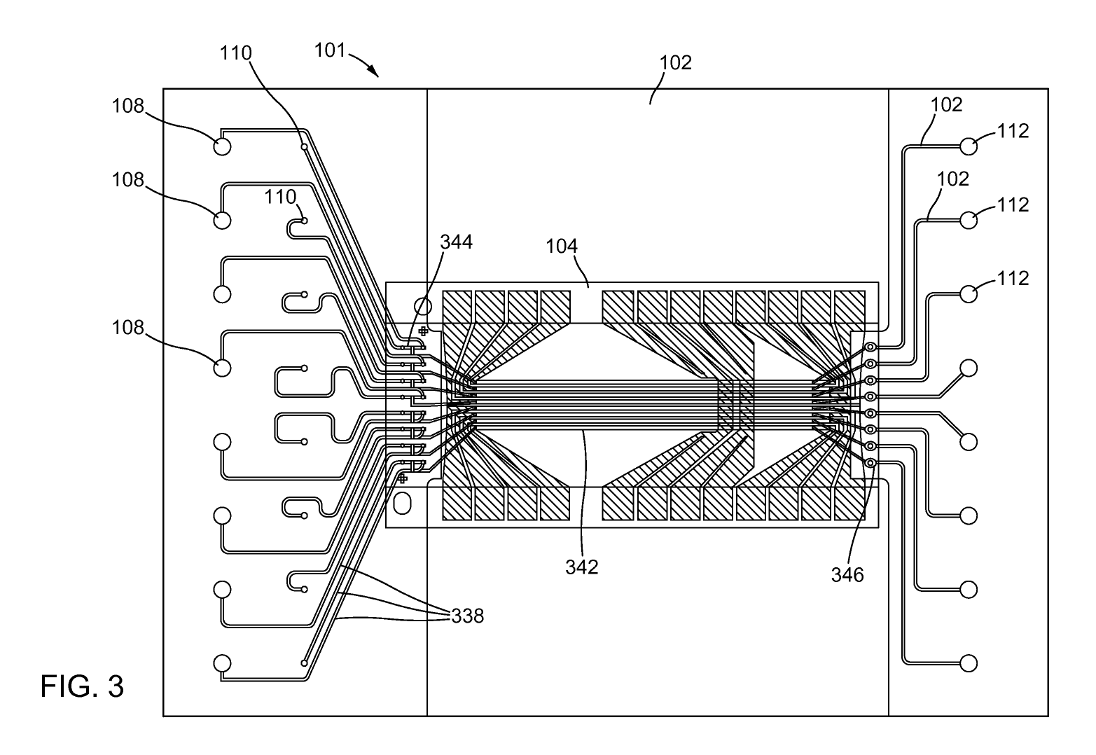

FIG. 3 illustrates a non-limiting example of a fluidic device 101 that includes an interface chip 102 and a reaction chip 104. In some embodiments, as illustrated in FIG. 3, the interface chip 102 may include a channel network that includes one or more input-side channels 338 and one or more output-side channels 340. In some embodiments, the reaction chip 104 may include a channel network that includes one or more channels 342. In some non-limiting embodiments, one or more of the channels 338, 340, and 342 may be microchannels. In some non-limiting embodiments, the channel network of the reaction chip 104 may also include one or more T-junctions 344. The input-side channels 338 may connect an inlet 110 to a first port of each of the one or more T-junctions 344, and the input-side channels 338 may connect a second port of each of the one or more T-junctions 344 to a vent well 108. In some embodiments, the reaction chip 104 may include one or more outlet ports 346, and the one or more channels 342 of the reaction chip 104 may extend from a T-junction 344 to an outlet port 346. Fluid may be controllably loaded into a channel 342 (or portion thereof) by using an outlet port 346 to pull fluid from a T-junction 344.

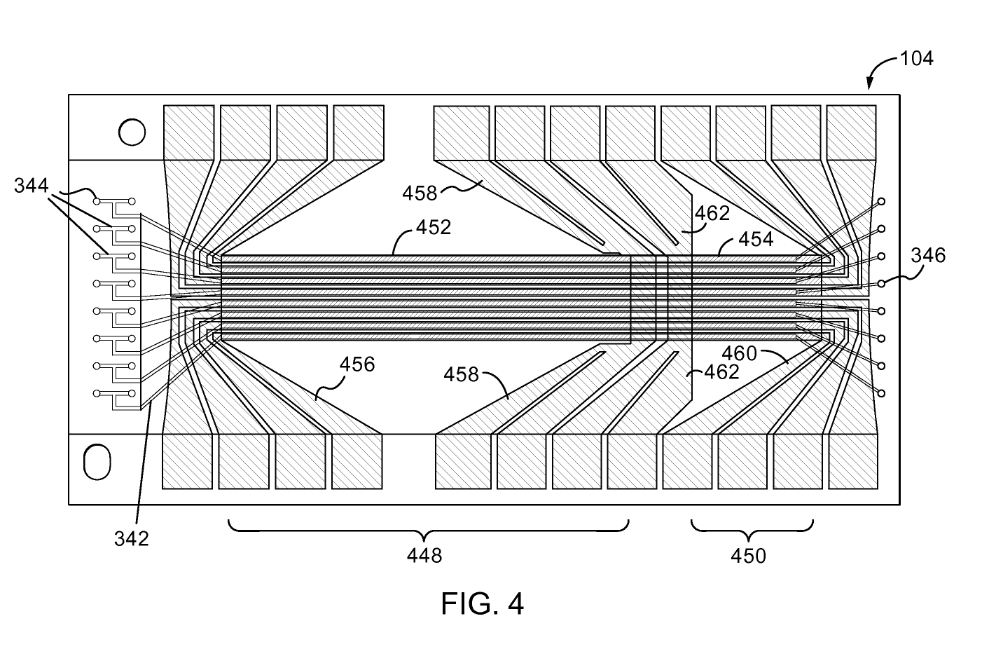

FIG. 4 illustrates a non-limiting example of a reaction chip 104. In some embodiments, as illustrated in FIG. 4, the reaction chip 104 may include one or more thermal zones 448 and 450 through which each of the one or more channels 342 may pass. In some non-limiting embodiments, the first thermal zone 448 may be a PCR thermal zone, and the second thermal zone 450 may be a thermal melt zone. In some embodiments, the reaction chip 104 may include one or more individually-controlled heater and/or sensor elements (e.g., resistive sensors) 452 associated with a first thermal zone 448. In some embodiments, the reaction chip 104 may include one or more individually-controlled heater and/or sensor elements (e.g., resistive sensors) 454 associated with a second thermal zone 450. In some non-limiting embodiments, one or more of the heater and/or sensor elements 452 and 454 may be in the form of a thin film resistive heater associated with a channel 342 in a thermal zone 448 or 450. The resistances of the thin film resistive heaters may be measured in order to control the respective temperatures of the thin film resistive heaters.

In some embodiments, electrodes 456 and 458 may provide power to the heater and/or sensor elements 452 (e.g., to cause PCR in fluid in channels 342 in the first thermal zone 448), and electrodes 460 and 462 may provide power to the heater and/or sensor elements 454 (e.g., to cause a thermal ramp in fluid in channels 342 in the second thermal zone 450). In some non-limiting embodiments, to utilize the limited space provided by the substrate of the reaction chip 104 and reduce the number of electrical connections required, multiple heater and/or sensor elements 452 may share one or more common electrodes 458, and multiple heater and/or sensor elements 454 may share one or more common electrodes 462.

In some embodiments, as illustrated in FIGS. 1 and 2, the LOC device 100 may include one or more flexible circuits 116 and 118 for electrical interconnection. In some non-limiting embodiments, the flexible circuits 116 and 118 may provide electrical access to the interface chip 102 and/or reaction chip 104 for communication of power and/or one or more control signals to the fluidic device 338 and/or communication of one or more measurement signals from the fluidic device 338. In some non-limiting embodiments, the LOC device 100 may include one or more keying and alignment features (e.g., the four holes in the layer 230 shown in FIG. 2, which line up with holes in the layers 224, 226, and 228) for positioning the LOC device 100 in a test apparatus and/or in an end-use instrument.

In some embodiments, as illustrated in FIGS. 1 and 2, the LOC device 100 may include one or more heat sinks 120 and 122 for thermal control. In some non-limiting embodiments, the heat sink 120 may be associated with a first (e.g., PCR) thermal zone 448, and the heat sink 122 may be associated with the second (e.g., thermal melt) thermal zone 450. In a non-limiting embodiment, as illustrated in FIGS. 1 and 2, the one or more heat sinks 120 and 122 may be pin-fin heat sinks having fins extending upwards from reaction chip 104 in a substantially vertical direction. However, this is not required, and some alternative embodiments may use other fin designs such as, for example and without limitation, straight, louvered, or bent fins.

In some embodiments, the interface chip 102 and/or reaction chip 104 may be built up from one or more layers of subcomponents (e.g., thin plastic layers or glass slides or chips). For example, in some non-limiting embodiments, as illustrated in FIG. 2, the interface chip 102 may be built up from one or more layers or subcomponents 224, 226, 228, 230, 232, and 234. In some non-limiting embodiments, the interface chip 102 and/or reaction chip 104 of the fluidic device 338 may be constructed using one or more of pressure sensitive adhesives, anodic bonding, thermal bond, glues, plastic welds, and epoxies. The bonding of various layers presents various possible failure modes. One possible failure mode is a leak between adjacent channels 338, 340, or 342 that are formed in the same layer. Another possible failure mode is a leak between adjacent wells or surface reservoirs 106 or 114 that are formed using the same layers. Leaks may develop because of hair-line cracks or delamination, which may be caused by, for example, loading stress, thermal shock, and/or thermal cycling. Delamination or voids where two layers are bonded may connect adjacent wells or reservoirs 106 or 114, adjacent vent wells 108, adjacent inlets 110, adjacent outlets 112, adjacent T-junction ports, and/or adjacent outlet ports 346.

For example, in an embodiment where a pressure sensitive adhesive layer is used to bond two plastic layers to form a series of wells or reservoirs (e.g., wells or reservoirs 106), delamination may cause one or more voids in the adhesive layer, and the one or more voids may connect two or more wells or reservoirs (e.g., two adjacent wells). For another example, in an embodiment where a pressure sensitive adhesive layer is used to bond two plastic layers to form a series of wells or reservoirs (e.g., wells or reservoirs 114) and outlets (e.g., outlets 112), delamination may cause one or more voids in the adhesive layer may connect two or more wells or reservoirs to each other, two or more outlets to each other, and/or one or more wells or reservoirs to one or more outlets.

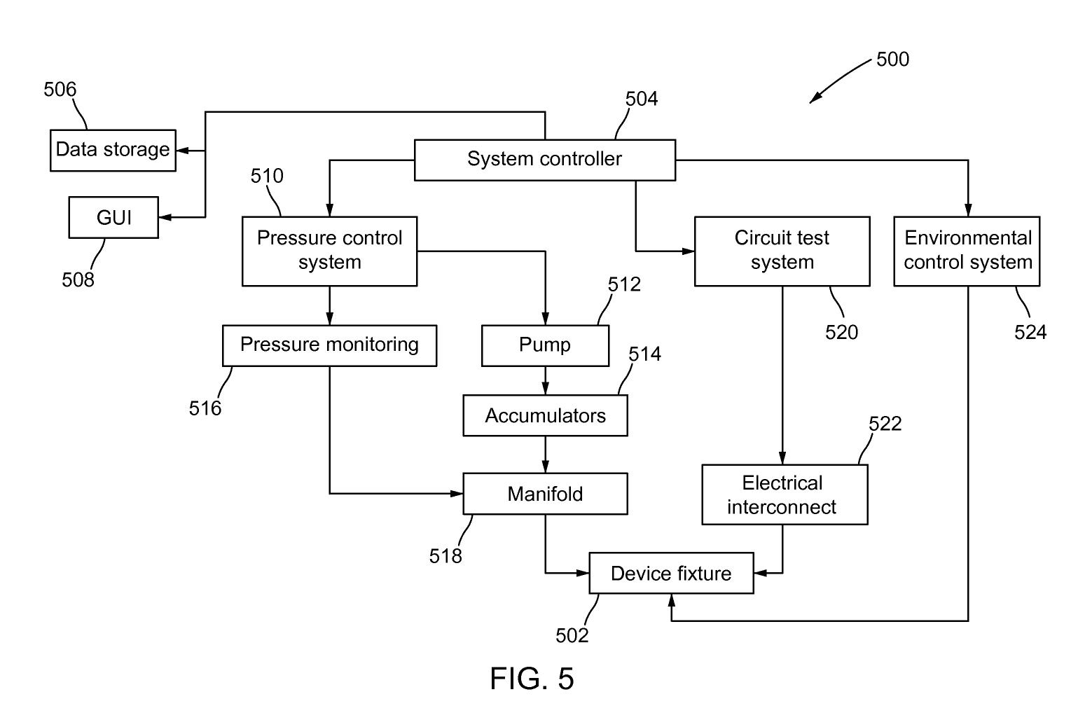

FIG. 5 is a schematic diagram illustrating an LOC test and/or burn-in system 500 embodying aspects of the present invention. In some embodiments, the system 500 may be configured to detect one or more possible failure modes for an LOC device (e.g., LOC device 100). In some embodiments, the system 500 may include a device fixture 502 into which an LOC device (or one or more components thereof) is placed for testing and/or burn-in. In some embodiments, the system 500 may include a system controller 504 to control testing of an LOC device. In some embodiments, the system controller 504 may store test results in a storage medium 506 (e.g., a non-transitory storage medium). In some embodiments, the system controller 504 may present test results (e.g., using a graphical user interface 508).

In some embodiments, the system 500 may include pressure control system 510 to test one or more fluidic features of a fluidic device (e.g., fluidic device 101) of an LOC device. In some embodiments, the system 500 may include one or more pumps 512, one or more valves (not shown), and/or one or more accumulators 514. The pressure control system 510 may control evacuating and/or pressurizing of one or more fluidic features and create one or more desired pressures using the one or more pumps 512, one or more valves, and/or one or more accumulators 514. In some embodiments, the system 500 may include one or more pressure monitors (e.g., pressure transducers) 516, and the pressure control system 510 may monitor the evacuating and/or pressurizing of the one or more fluidic features using the one or more pressure monitors 516. In some embodiments, the system 500 may include a manifold 518, and pressure control may be interfaced with the LOC device via the manifold 518.

In some embodiments, when the LOC device 100 illustrated in FIGS. 1-4 (or fluidic device 101 thereof) is loaded into the LOC test and/or burn-in system 500, the system 500 may be configured to create a closed volume and monitor pressure at the vent wells or outlets 108, apply positive or negative pressure to the sipper wells or inlets 110, and create a close volume and monitor pressure at one or more of the waste wells or inlets 112. In some non-limiting embodiments, the system 500 may test one or more networks of the fluidic device 101, which may each include channels 338, 340, and 342, a T-junction 344, a sipper well 110, a vent well 108, and a waste well 112 (as illustrated in FIG. 3), to determine whether the network is sealed and not cross-connected.

In some embodiments, the system 500 may include a circuit test system 520 configured to test one or more electrical features of the LOC device. The circuit test system 520 may interface with the LOC device via electrical interconnects 522. In some embodiments, the system 500 may additionally or alternatively include an environmental control system 524 that controls one or more environmental conditions under which the test is performed. In some non-limiting embodiments, the environmental control system 524 may include, for example, one or more of a temperature measurement and control device, a static pressure measurement and control device, and a humidity measurement and control device.

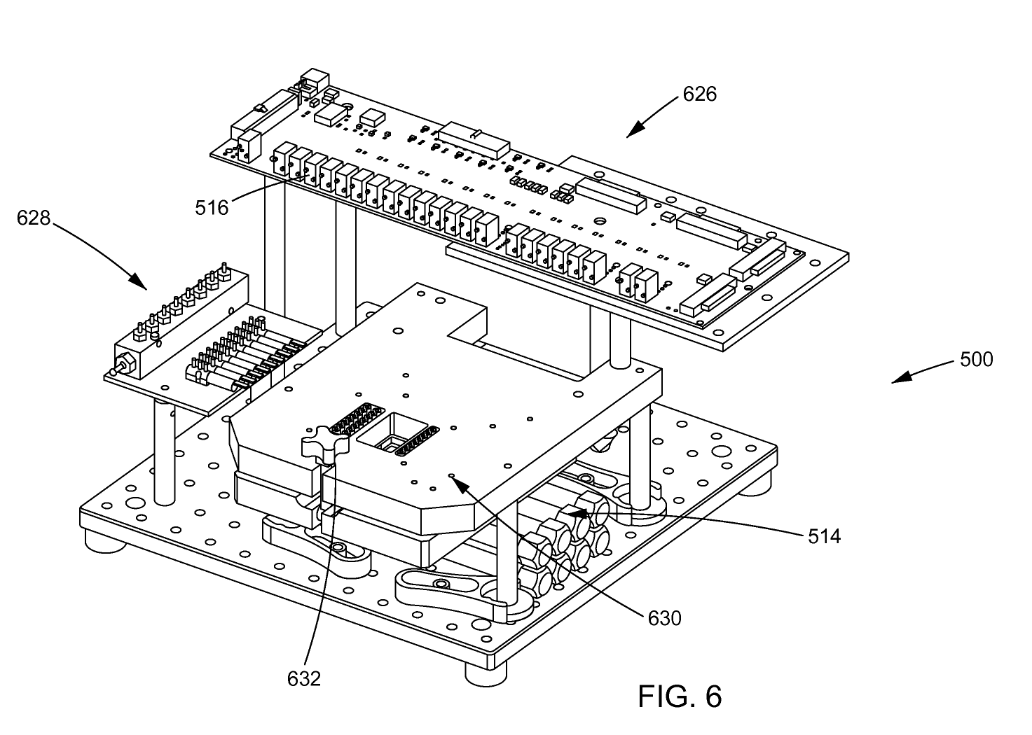

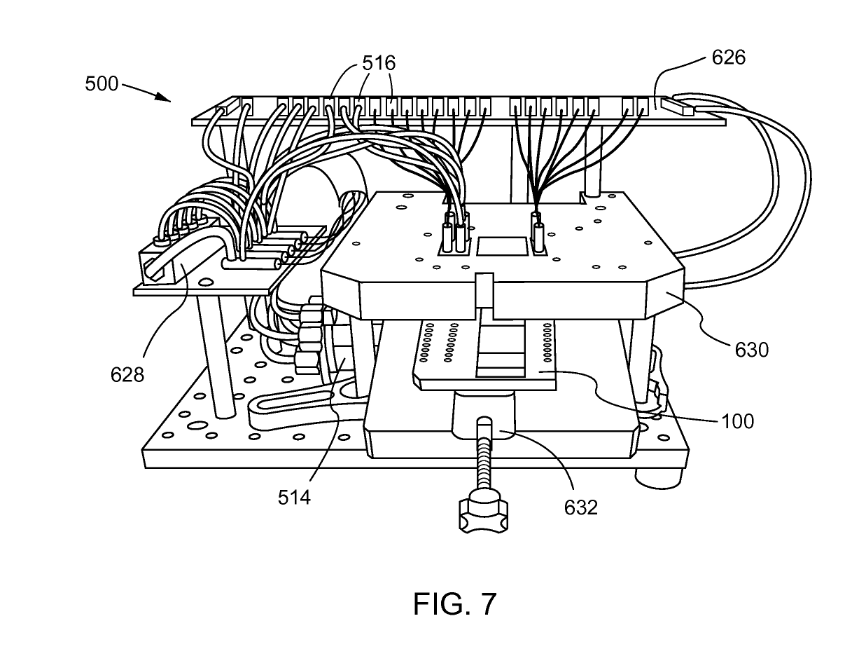

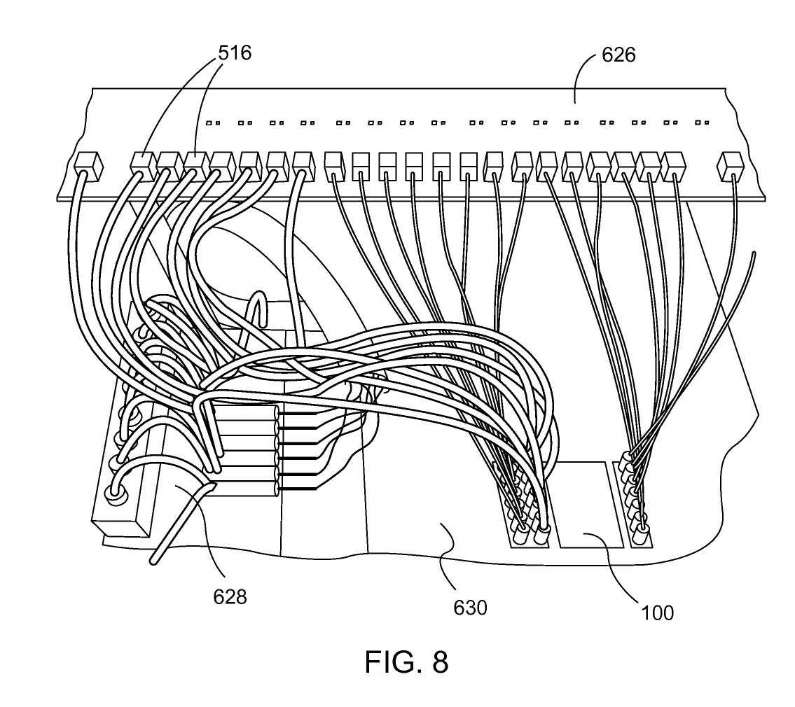

FIGS. 6-8 illustrate an LOC test and/or burn-in system 500 embodying aspects of the present invention. FIG. 6 is a perspective view of a top, rear, and left side of a closed system 500 according to some embodiments. FIG. 7 is a perspective view of a top and left side of an open system 500 according to some embodiments. FIG. 8 is a perspective view of a portion of a top and left side of the system 500 and illustrates fluidic and electrical connections in the system 500.

In some non-limiting embodiments, as illustrated in FIGS. 6-8, the system 500 may include a control and measurement printed circuit board (PCB) 626. In some embodiments, the control and measurement PCB 626 may incorporate one or more of the pressure measurement functionality of the one or more pressure monitors 516, the electrical measurement functionality of the circuit test system 520, the valve control functionality of the pressure control system 510, and the environmental control functionality of the environmental control system 524. The control and measurement PCB 626 may also be configured to interface with the system controller 504.

In some embodiments, as illustrated in FIGS. 6-8, the LOC test and/or burn-in system 500 may include a pressure control module 628 configured to control one or more valves of the system 500 to evacuate (i.e., apply a vacuum or a negative pressure to) one or more fluidic features of the LOC device 100 and/or to pressurize (i.e., apply a positive pressure to) one or more fluidic features of the LOC device 100.

In some embodiments, as illustrated in FIGS. 6-8, the LOC test and/or burn-in system 500 may include a device interface module 630. In some embodiments, the device interface module 630 may include one or more of the device fixture 502, manifold 518, and electrical interconnect 522 shown in FIG. 5. In some embodiments, the device interface module 630 may hold the device 100 during testing and/or burn-in. In some embodiments, the device interface module 630 may make pressure connections and/or electrical connections to the LOC device 100. As illustrated in FIGS. 6-8, in some embodiments, the one or more pressure monitors 516 may be connected to the device interface module 630 via one or more tubing elements.

In some embodiments, as illustrated in FIGS. 7 and 8, the device interface module 630 may include a screw clamp 632 for loading of an LOC device 100 into the device interface module 630. FIG. 6 shows the screw clamp 632 holding the device interface module 630 in its closed position, and FIG. 7 shows an unlatched screw clamp 632 with the device interface module 630 in its open position. Although, in some embodiments of the system 500, the device interface module 630 may include a screw clamp 632, this is not required. In some alternative embodiments, loading of an LOC device 100 into the device interface module 630 could be accomplished by other means, such as, for example and without limitation, a different clamp or latch. Moreover, in some alternative embodiments, loading of an LOC device 100 into the device interface module 630 could additionally or alternatively be accomplished using one or more of robotic, pneumatic (e.g. vacuum chuck), and electromagnetic actuation.

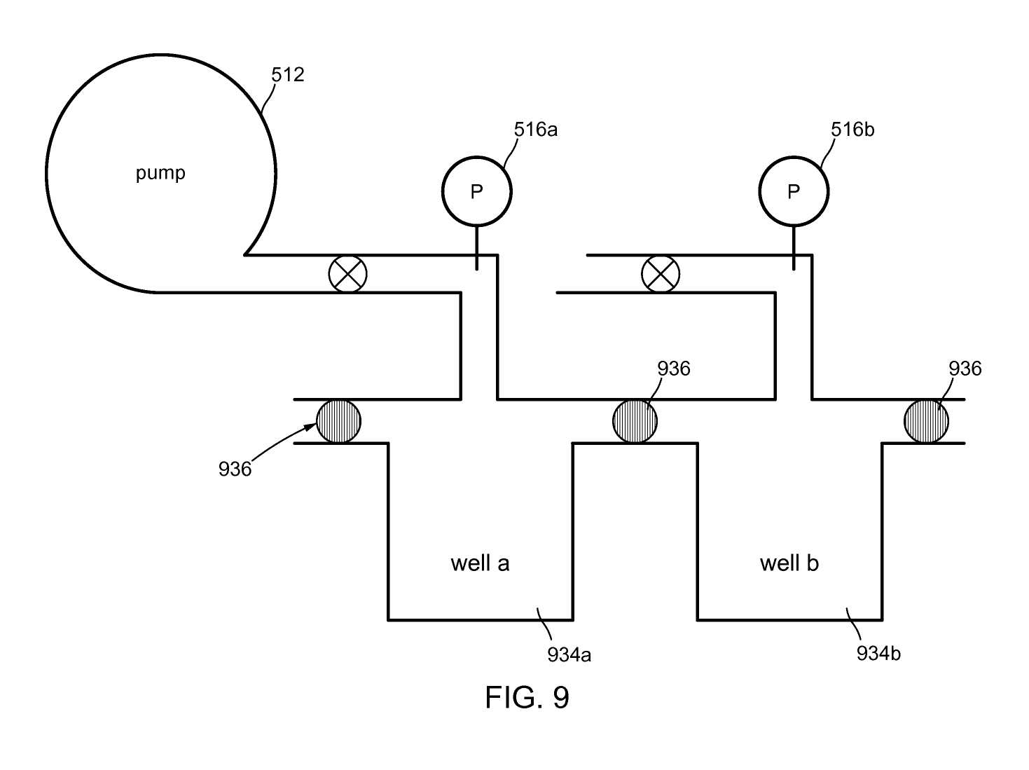

FIG. 9 is a schematic diagram illustrating an LOC test and/or burn-in system 500 configured to test an LOC device 100 according to some embodiments of the invention. As illustrated in FIG. 9, the LOC device 100 may include fluidic features 934a and 934b. In some non-limiting embodiments, the fluidic features 934a and 934b may, for example and without limitation, correspond to wells or reservoirs 106, vent wells 108, inlets 110, outlets 112, or wells or reservoirs 114. In some embodiments, the LOC device 100 may include one or more additional fluidic features. In the illustrated embodiment, the fluidic features 934a and 934b are wells (e.g., wells 106). However, this is not required, and, in some alternative embodiments, one or more of the fluidic features 934a and 934b may be a different type of fluidic feature, such as, for example and without limitation, a buried reservoir, a surface reservoir, a reaction chamber, a channel (e.g., a microchannel), or a channel network (e.g., a microchannel network). In some embodiments, the fluidic features 934 may be separated by one or more gaskets 936.

As illustrated in FIG. 9, the fluidic feature 934a may be connected to a pump 512 of the system 500. In some embodiments, the pump 512 may be configured to evacuate or pressurize the fluidic feature 934a. In some embodiments, a pressure monitor 516a may be configured to measure a pressure response of the fluidic feature 934a. In some embodiments, a pressure monitor 516b may be configured to alternatively or additionally measure a pressure response of the fluidic feature 934b.

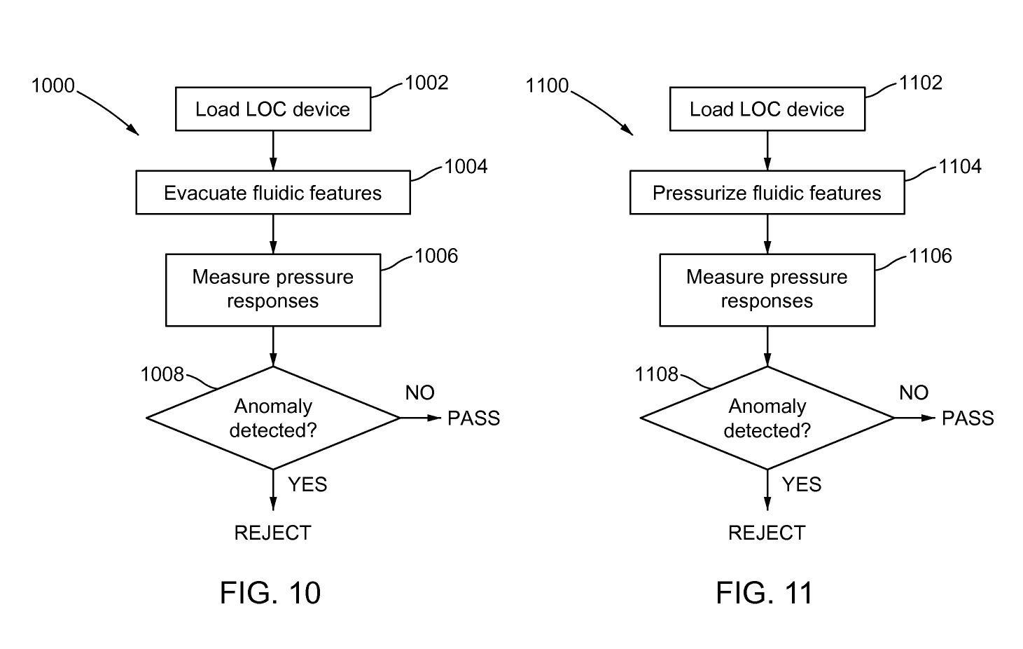

FIG. 10 is a flowchart illustrating a process 1000 for testing one or more fluidic features (e.g., one or more wells or reservoirs 106 and 114, one or more vent wells 108, one or more inlets 110, one or more outlets 112, one or more channels 338, one or more channels 340, and/or one or more channels 342) using an LOC test and/or burn-in system 500. In some embodiments, the process 1000 may include a step 1002 of loading an LOC device (e.g., LOC device 100) or a component thereof (e.g., fluidic device 101) into the system 500. In some non-limiting embodiments, the step 1002 may include loading the LOC device into the device interface module 630 of the system 500.

In some embodiments, the process 1000 may include a step 1004 of evacuating one or more fluidic features of the LOC device (e.g., by applying a vacuum or negative differential pressure to the one or more fluidic features of the LOC device). In some embodiments, the process 1000 may include a step 1006 of monitoring a pressure response of the one or more evacuated fluidic features. Monitoring the one or more evacuated fluidic features may allow the system 500 to detect leaks and blockages of the evacuated fluidic features. In some non-limiting embodiments, the step 1006 may include monitoring a pressure response of one or more fluidic features that were not evacuated in step 1004 (e.g., monitoring the pressure response of one or more fluidic features surrounding the one or more evacuated fluidic features). For instance, in one non-limiting embodiment, the process 1000 may evacuate fluidic feature 934a in step 1004 and monitor the pressure response of fluidic features 934a and 934b in step 1006. Monitoring one or more surrounding features may allow the system 500 to detect cross-connection of fluidic features to be determined.

In some embodiments, the process 1000 may include a step 1008 of detecting anomalies, such as, for example and without limitation, leaks, blockages, and/or cross-connection. Leaks may be evident if one or more evacuated fluidic features do not reach the desired negative pressure or are incapable of holding the negative pressure. Blockages are evident if one or more of the evacuated fluidic features do not reach the desired negative pressure. Cross-contamination is evident if evacuating a fluidic feature changes the pressure in one or more surrounding fluidic features. Leaks, blockages, and cross-connection may all be modes of device failure. These modes would likely present themselves early (even immediately) upon LOC device usage making the LOC device a so called “infant mortality” failures. If any of these anomalies are detected during test/burn-in, then the LOC device (or component thereof) could be rejected, preventing failure of the LOC device during normal use.

FIG. 11 is a flowchart illustrating a process 1100 for testing one or more fluidic features using an LOC test and/or burn-in system 500. In some embodiments, the process 1100 may include a step 1102 of loading an LOC device (e.g., LOC device 100) or a component thereof (e.g., fluidic device 101) into the system 500. In some embodiments, the process 1100 may include a step 1104 of pressurizing one or more fluidic features of the LOC device (e.g., by applying a positive differential pressure to the one or more fluidic features of the LOC device). In some embodiments, the process 1100 may include a step 1106 of monitoring a pressure response of the one or more pressurized fluidic features. Monitoring the one or more pressurized fluidic features may allow the system 500 to detect leaks and blockages of the pressurized fluidic features. In some non-limiting embodiments, the step 1106 may include monitoring a pressure response of one or more fluidic features that were not pressurized in step 1104 (e.g., monitoring the pressure response of one or more fluidic features surrounding the one or more pressurized fluidic features). For instance, in one non-limiting embodiment, the process 1100 may pressurize fluidic feature 934a in step 1104 and monitor the pressure response of fluidic features 934a and 934b in step 1106. Monitoring one or more surrounding features may allow the system 500 to detect cross-connection of fluidic features to be determined.

In some embodiments, the process 1100 may include a step 1108 of detecting anomalies, such as, for example and without limitation, leaks, blockages, and/or cross-connection. Leaks may be evident if one or more pressurized fluidic features do not reach the desired positive pressure or are incapable of holding the positive pressure. Blockages are evident if one or more of the pressurized fluidic features do not reach the desired positive pressure. Cross-contamination is evident if evacuating a fluidic feature changes the pressure in one or more surrounding fluidic features. Leaks, blockages, and cross-connection may all be modes of device failure. If any of these anomalies are detected during test/burn-in, then the LOC device (or component thereof) could be rejected, preventing failure of the LOC device during normal use.

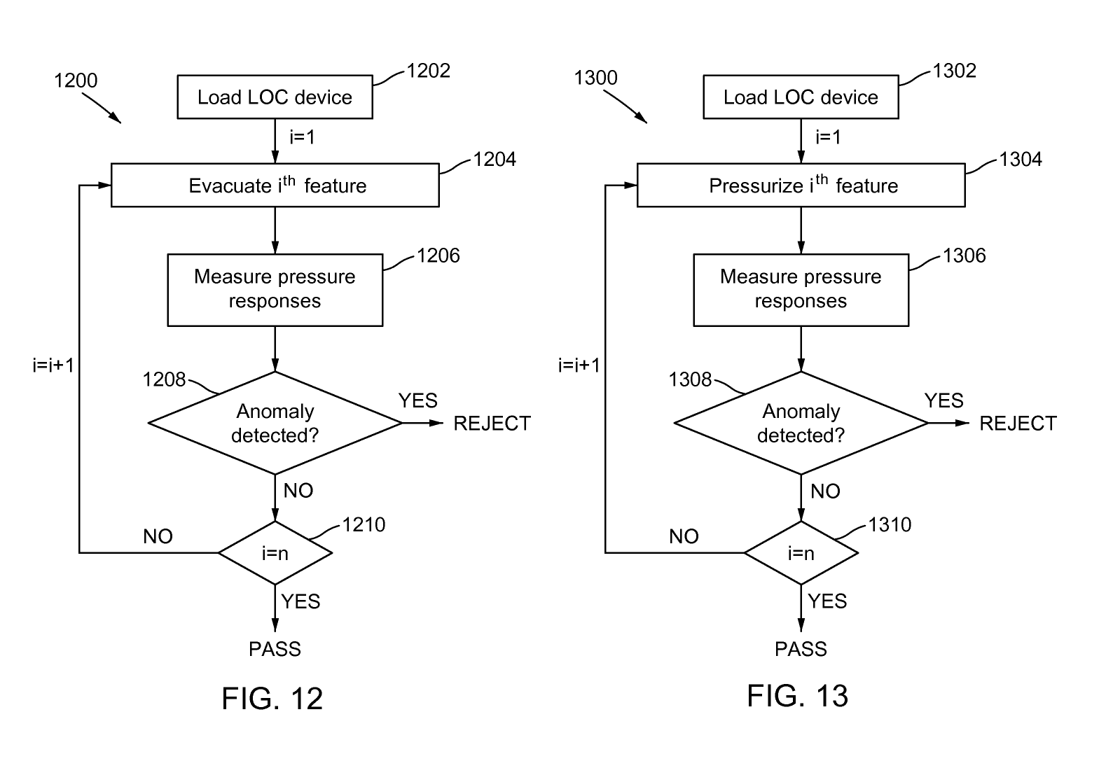

FIG. 12 is a flowchart illustrating a process 1200 for individually testing n fluidic features of an LOC device. In some embodiments, the process 1200 may include a step 1202 of loading an LOC device (e.g., LOC device 100) or a component thereof (e.g., fluidic device 101) into the system 500. In some embodiments, the process 1200 may include a step 1204 of evacuating an i^{th} fluidic feature. In some embodiments, the process 1200 may include a step 1206 of monitoring a pressure response of the i^{th} fluidic feature and/or one or more other fluidic features. In some non-limiting embodiments, the process 1200 may include a step 1208 of detecting anomalies. If an anomaly is detected, the system 500 may reject the loaded LOC device (or component thereof). In some embodiments, the process 1200 may include a step 1210 in which the system 500 determines whether i=n. In some embodiments, the process 1200 may proceed to step 1210 if no anomalies are detected in step 1208. If the system 500 determines that inn, the system 500 may increment i and then loop back to step 1204. If the system 500 determines that i=n, the system 500 may determine that the LOC device (or component thereof) has passed the test.

FIG. 13 is a flowchart illustrating a process 1300 for individually testing n fluidic features of an LOC device. In some embodiments, the process 1300 may include a step 1302 of loading an LOC device (e.g., LOC device 100) or a component thereof (e.g., fluidic device 101) into the system 500. In some embodiments, the process 1300 may include a step 1304 of pressurizing an i^{th} fluidic feature. In some embodiments, the process 1300 may include a step 1306 of monitoring a pressure response of the i^{th} fluidic feature and/or one or more other fluidic features. In some non-limiting embodiments, the process 1300 may include a step 1308 of detecting anomalies. If an anomaly is detected, the system 500 may reject the loaded LOC device (or component thereof). In some embodiments, the process 1300 may include a step 1310 in which the system 500 determines whether i=n. In some embodiments, the process 1300 may proceed to step 1310 if no anomalies are detected in step 1308. If the system 500 determines that i≠n, the system 500 may increment i and then loop back to step 1304. If the system 500 determines that i=n, the system 500 may determine that the LOC device (or component thereof) has passed the test.

The processes 1200 and 1300 in which n fluidic features are individually tested may be particularly useful for detection of cross-connection to adjacent features. For example, if the process 1200 or 1300 were used to test an LOC device 100 having an interface chip 102 with voids in an adhesive layer that connect two adjacent sample wells 106, the system 500 may be used to detect a leak in one or both of the connected sample wells 106 (e.g., by detecting that one or both of the connected sample wells 106 do not reach the desired pressure or are incapable of holding the pressure) and/or the cross-connection (e.g., by detecting a change in pressure in one of the connected sample wells 106 when the other of the connected sample wells 106 is evacuated or pressurized), and the system 500 may reject the LOC device 100 having the voids in the interface chip 102.

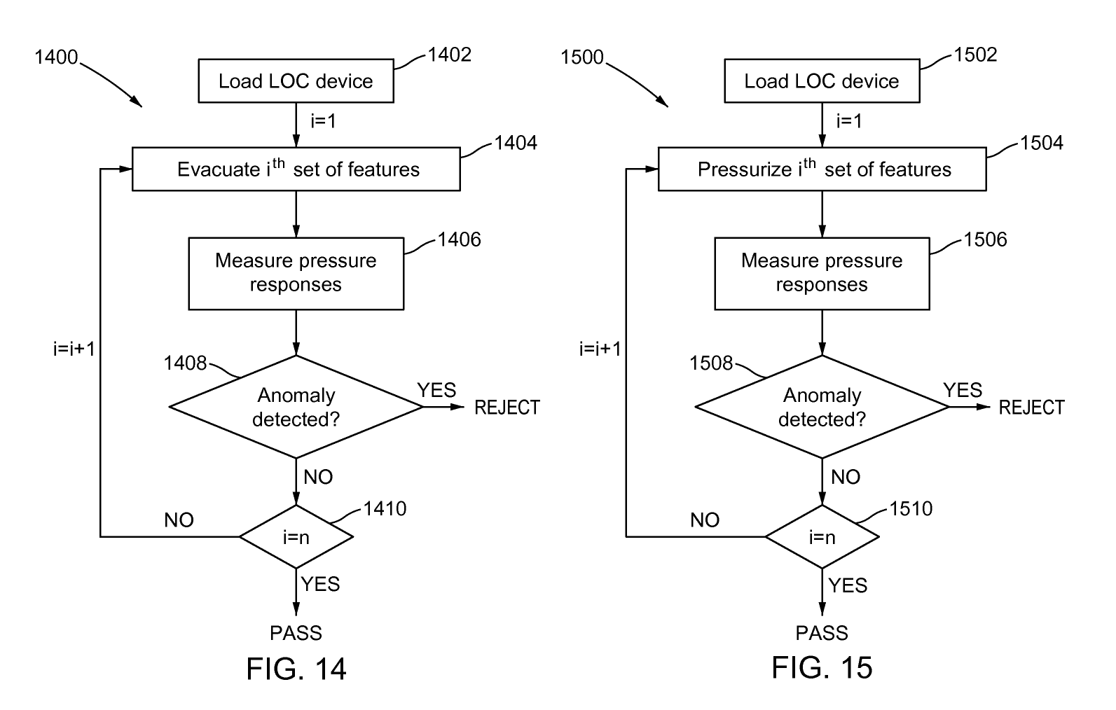

Although processes 1200 and 1300 are linear iterative processes, any order of test could be used. For example, FIG. 14 is a flowchart illustrating a process 1400 for individually testing n sets of fluidic features of an LOC device. In some embodiments, one or more of the n sets of fluidic features may include multiple fluidic features. In some embodiments, the process 1400 may include a step 1402 of loading an LOC device (e.g., LOC device 100) or a component thereof (e.g., fluidic device 101) into the system 500. In some embodiments, the process 1400 may include a step 1404 of evacuating an i^{th} set of fluidic features. In some embodiments, the process 1400 may include a step 1406 of monitoring a pressure response of the i^{th} set of fluidic features and/or one or more fluidic features not in the i^{th} set of fluidic features. In some non-limiting embodiments, the process 1400 may include a step 1408 of detecting anomalies. If an anomaly is detected, the system 500 may reject the loaded LOC device (or component thereof). In some embodiments, the process 1400 may include a step 1410 in which the system 500 determines whether i=n. In some embodiments, the process 1400 may proceed to step 1410 if no anomalies are detected in step 1408. If the system 500 determines that i≠n, the system 500 may increment i and then loop back to step 1404. If the system 500 determines that i=n, the system 500 may determine that the LOC device (or component thereof) has passed the test.

FIG. 15 is a flowchart illustrating a process 1500 for individually testing n sets of fluidic features of an LOC device. In some embodiments, one or more of the n sets of fluidic features may include multiple fluidic features. In some embodiments, the process 1500 may include steps 1502, 1504, 1506, 1508, and 1510 that correspond to steps 1402, 1404, 1406, 1408, and 1410, respectively, except that in step 1504 the i^{th} set of fluidic features is pressurized instead of evacuated. In some alternative embodiments, the step 1504 may include evacuating one or more fluidic features of the i^{th} set and pressurizing one or more fluidic features of the i^{th} set.

In some non-limiting embodiments, the processes 1400 or 1500 could be used to, for example and without limitation, evacuate or pressurize odd numbered channels (e.g., odd-numbered channels 338, 340, and and/or 342 of FIG. 3) while even numbered channels are monitored and vice versa.

In some embodiments, the system controller 504 may save (e.g., in storage medium 506) the pressure response data measured during the testing (e.g., measured in any of the processes 1000, 1100, 1200, 1300, 1400, and 1500) as a calibration of the device, which may be used by the end-user

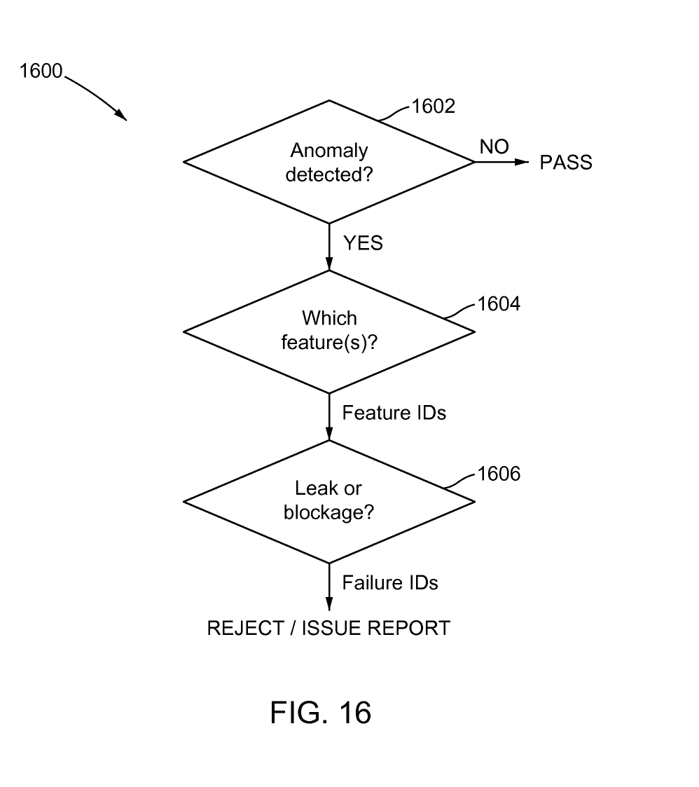

In some embodiments, one or more of the processes 1000, 1100, 1200, 1300, 1400, and 1500 could be carried out under the control of the system controller 504 of the LOC test and/or burn-in system 500. In some non-limiting embodiments, the system controller 504 may make an accounting of the failures that are detected. In some non-limiting embodiments, the system controller 504 may use the accounting of failures to create test reports, which may be used for quality assurance or as the beginning to a re-work process in which the part is re-worked so that it can be used in the future (e.g., re-pressing components to improve a bond or re-attachment of electrical connectors). FIG. 16 is a flowchart illustrating a process 1600 for issuing a test report according to some non-limiting embodiments. In some embodiments, the process 1600 may include a step 1602 of determining whether an anomaly has been detected (e.g., in any of steps 1008, 1108, 1208, 1308, 1408, or 1508 of FIGS. 10-15). In some embodiments, the process 1600 may include a step 1604 of identifying which fluidic feature(s) generated the anomaly. In some embodiments, the process 1600 may include a step 1606 of identifying whether the anomaly is a leak or a blockage. The system controller 504 may then issue a test report identifying the defective fluidic feature(s) and the type of defect.

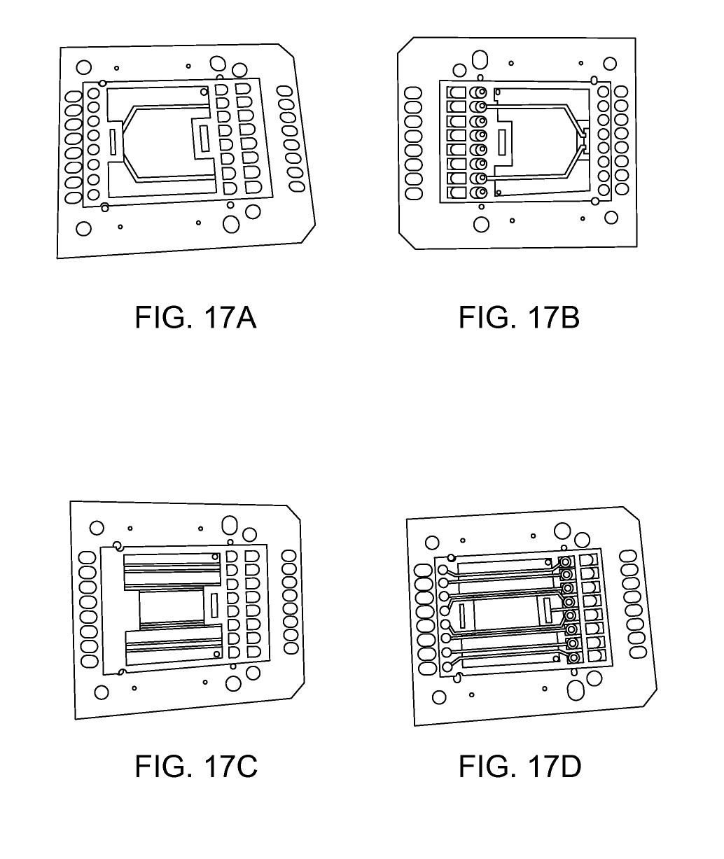

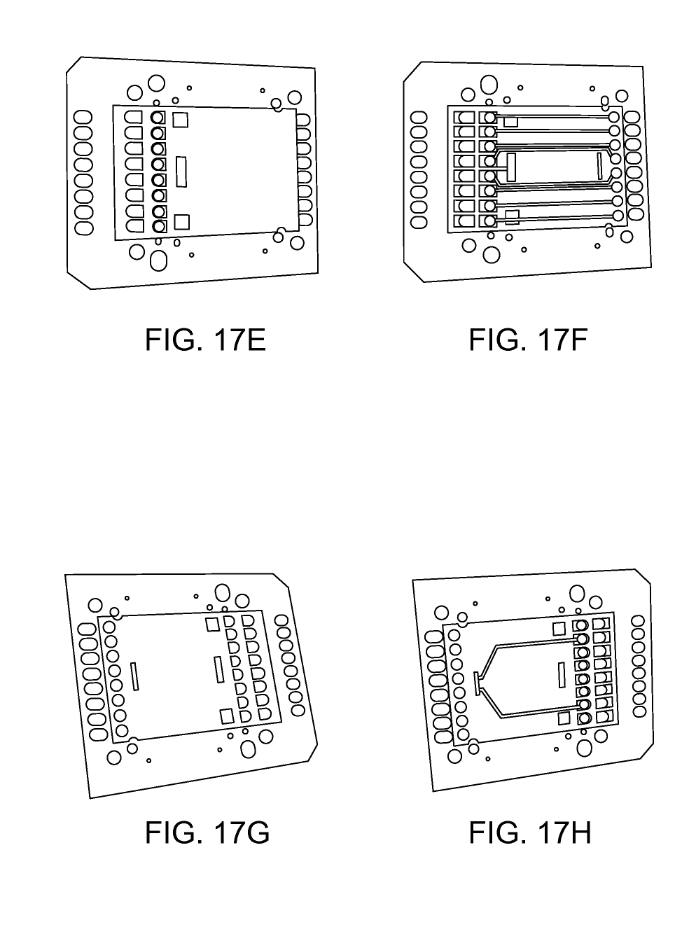

As mentioned above, individual components of an LOC device may be tested using the processes 1000, 1100, 1200, 1300, 1400, and 1500. Testing individual components may improve device yield as relatively simple components can be tested individually before the device is fully assembled. In some embodiments, the system 500 may include different component testing fixtures to adapt pressure and/or electrical connections to the different individual components. That is, the device fixture 502 of the system 500 may be different depending on whether the system 500 is testing an LOC device or a component thereof and/or depending on which component the system 500 is testing. FIGS. 17A-17H illustrate non-limiting examples of different device fixtures that may be used to test different LOC device components. The gaskets in the adaptors illustrated in FIGS. 17A-17H may plug some fluidic ports and re-pipe some connections to allow fluidic features with different configurations to be tested in the same test/burn-in system 500 used to test a complete LOC device. That is, each of the adaptors illustrated in FIGS. 17A-17H may allow a different component to be tested in the system 500. Although some embodiments use different device fixtures to adapt a system 500 to test different components, this is not required. In some alternative systems, a separate system 500 could be developed for each component.

In some embodiments, the circuit test system 520 of the LOC test and/or burn-in system 500 may test one or more electrical features of a loaded LOC device (or component thereof) at the same time as or serially with testing one or more fluidic features. In some embodiments, an electrical feature may be, for example and without limitations, a heater, a sensor, a resistor, a capacitor, a controller, a counter, a timer, memory, a processor, an actuator, a valve, or another feature known in the art. In some embodiments, the system 500 may determine whether one or more electrical features of an LOC device meet certain specifications to determine whether the LOC device passes or fails. For example, the system 500 may determine whether a resistance value falls within a specified range or whether a processor is capable of performing a fixed set of calculations within a specified period of time.

In some embodiments, the system 500 may power one or more electrical features of a loaded LOC device (e.g., LOC device 100) to run a pre-determined program for a burn-in test. In some non-limiting embodiments, the burn-in test may consist of using heating elements (e.g., thin film resistive heaters) of the loaded LOC device. In some embodiments, the system 500 may use one or more heating elements at their normal operating conditions or at higher than normal conditions to accelerate the test. Running at higher or harsher conditions may accelerate the test because certain failure modes may occur earlier at high temperatures, allowing these failures to be detected during burn-in rather than device usage by the end user. In some alternative embodiments, the system 500 may alternatively or additionally run a processor or controller of the loaded LOC device under a pre-determined program. The pre-program for the processor or controller could also simulate normal device usage or accelerate the test by running a more demanding program (e.g., more parallel processing or higher duty cycles). Again, in some embodiments, these tests/burn-in may be run at the same time or serially with the fluidic feature testing.

In some embodiments where the loaded LOC device is configured to perform a polymerase chain reaction (PCR), the system 500 may perform a burn-in test that includes cycling the LOC device through the typical PCR temperatures for the normal PCR times. In some non-limiting embodiments, the system 500 could cause the LOC device to perform a fixed number of PCR cycles (e.g., 40 cycles, which is approximately one amplification experiment, or 400 cycles, which is approximately ten amplification experiments). In some embodiments, the system 500 may test one or more fluidic features and/or one or more other electrical features of the LOC device during and/or after the PCR cycling. In this manner, the system 500 may qualify the device (e.g., if the system 500 determines that fluidic features of an LOC device are leak and blockage free after the LOC device runs a pre-determined number of PCR cycles, then the LOC device is ready for the end user who will also use the LOC device for PCR).

In some embodiments where the loaded LOC device is configured to perform sample preparation by heating fluid reservoirs or microchannels, the system 500 may similarly burn-in the LOC device by testing one or more fluidic features during and/or after tests that simulate the desired device usage (e.g., heating fluid to specific temperatures for specific times). In some embodiments where the loaded LOC device is configured to perform melt analysis, the system 500 may perform burn-in by simulating the desired device usage (e.g., genotyping or heating one or more samples to determine their melting characteristics).

In some embodiments, the system 500 may conduct one or more burn-in tests, for example, at one or more of an elevated temperature, an elevated static pressure, and/or an elevated relative humidity (RH). In some embodiments, environmental controls (e.g., environmental control system 524) may be built into the test/burn-in system 500 or alternatively the system 500 may be placed within an environmental chamber. In some embodiments, the harsher environmental conditions may accelerate testing by increasing the rate of device failure and allow the test of the LOC device to be completed in a shorter time. Harsher conditions may also provide a degree of conservatism (i.e., safety margin) to better qualify the components/device for service. For example, in one non-limiting embodiment, the system 500 may burn-in an LOC device that will only be used at room temperature (e.g., approximately 23 deg. C) and a maximum relative humidity of 50% at a higher temperature (e.g., approximately 30 deg. C) and/or at a higher relative humidity (e.g, 90%).

In some embodiments, the system 500 may cycle the LOC device through one or more temperatures or follow a specific pre-determined thermal profile. In some embodiments, subjecting the LOC device to a thermal profile during the test/burn-in of one or more fluidic and electrical features may to accelerate the test and may be used to test whether an LOC device can withstand shipping/storage conditions.

In some embodiments, the system 500 may subject one or more fluidic features of an LOC device to higher than normal pressures to test the integrity of the one or more fluidic features and the strength of the LOC device. For example, in one non-limiting embodiment, the system 500 may subject a feature that will normally be subjected to modest positive pressure differential (e.g., 1 psi above ambient (14.7 psi)) to a higher pressure differential (e.g., 2 psi above ambient). The higher pressure may put the one or more features and/or the LOC device under more stress and may cause failure, which could be detected during test/burn-in and is preferred to the failure occurring during device shipment or usage. In the example above, the system 500 may validate the LOC device to a safety factor of 2. However, other safety factors may alternatively be appropriate (e.g., a safety factor of 10, which would require the fluidic feature in the example above to be pressurized to 10 psi).

In some embodiments, the system 500 may test/burn-in fluidic features that may be used only under negative pressure differential conditions under positive pressure conditions. For example, placing a sub-surface reaction chamber under positive pressure (with respect to ambient) would place the LOC device under tensile stress as the pressure would tend to pull the device apart. Such a test would be a good test of the strength of the LOC device (more specifically the layers holding the LOC device together). For example, the system 500 may pressurize a reaction chamber to 11 atm to prove that the LOC device can withstand a 10 atm differential. The exact values used to determine strength would be device dependent. Further, any fluidic feature could be subjected to this test in addition to the sub-surface reaction chamber described above.

In some embodiments, the system 500 may dry test the LOC device using gases (e.g., air or nitrogen). In some alternative embodiments, the system 500 may test a portion or all of the LOC device wet (e.g., using water, alcohol, buffer solutions, solutions containing dye, etc.). In some embodiments, the pressure control system 510 controls one or more of the wet and dry testing. In some embodiments, the system 500 may use one or more pumps 512 and one or more pressure monitors 516 to load the fluid (in this case liquid) into one or more fluidic features. Wet testing may detect one or more failure modes that may not occur during dry tests alone. For example, materials of the LOC device may absorb water or other solvents affecting their performance (e.g., strength and/or sealing characteristics).

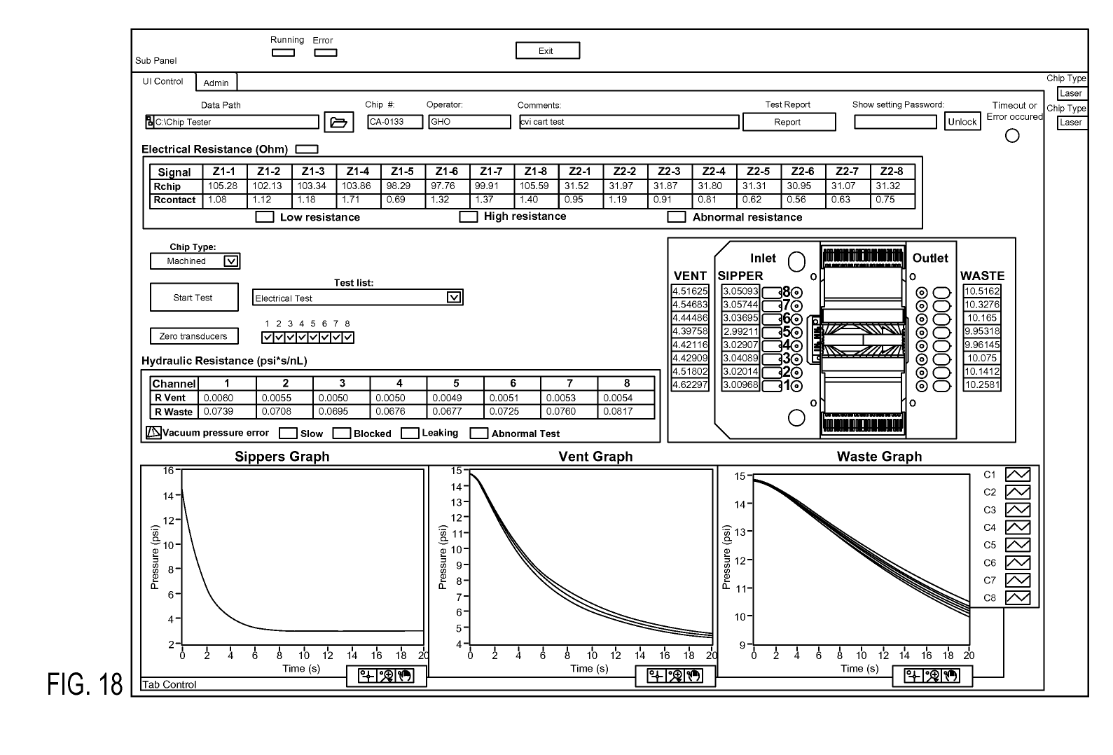

FIG. 18 is a screenshot of test results that may be presented by the system controller 502 of the LOC test and/or burn-in system 500 (e.g., using a graphical user interface 508) according to some embodiments of the present invention. In the example illustrated in FIG. 18, 32 electrical resistances are tested to determine whether the electrical resistances are within predetermined ranges at the same time as fluidic features are tested. In the example, the fluidic features are tested by evacuating 8 channel networks (e.g., the 8 channel networks including channels 338, 340, and and/or 342 illustrated in FIG. 3). The system 500 monitors the pressures at 24 inlet/outlet ports to determine whether the channels are blocked or leaking.

In some embodiments, the system 500 may additionally or alternatively perform proof testing on one or more fluidic features of a loaded LOC device. A proof test is a form of stress test to demonstrate the fitness of a load-bearing structure. An individual proof test may apply only to the unit (e.g., a fluidic feature) tested, or to its design in general for mass-produced items. A proof test may subject a structure to loads above that expected in actual use, thereby demonstrating safety and design margin. Proof testing may be nominally a nondestructive test, particularly if both design margins and test levels are well-chosen. However, unit failures may be considered to have been destroyed for their originally-intended use and load levels. In some embodiments, the system 500 may perform one or more proof tests before a new LOC device design or unit is allowed to enter service, or perform additional uses, or to verify that an existing unit is still functional as intended. In some embodiments, the system 500 may perform one or more proof tests to determine that one or more fluidic features are sealed (i.e., do not leak) and are not cross-connected.

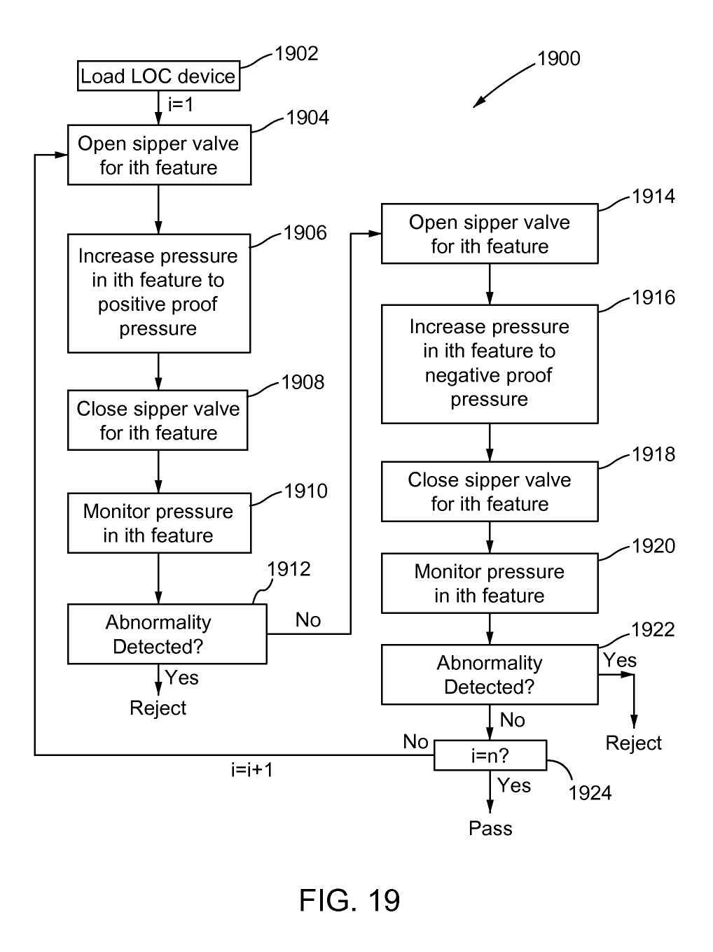

FIG. 19 is a flow chart illustrating a proof testing process 1900 according to some embodiments of the present invention. In some embodiments, the process 1900 may include a step 1902 of loading an LOC device (e.g., LOC device 100) or a component thereof (e.g., fluidic device 101) into the system 500. In some embodiments, the process 1900 may include a step 1904 of opening a valve in communication with an i^{th} fluidic feature. In some non-limiting embodiments, the valve may be a sipper valve, and the i^{th} fluidic feature may be an i^{th} channel (e.g., a channel 338. 342, and/or 340). In some embodiments, as illustrated in FIG. 3, the channel may be in communication with a waste well 112 and a vent well 108. In some non-limiting embodiments, the process 1900 may include a step 1906 of increasing pressure in the i^{th} fluidic feature to a positive proof pressure (e.g., +2 psig). In some embodiments, the process 1900 may include a step 1908 of closing the valve. In some embodiments, the process 1900 may include a step 1910 of monitoring pressure in the i^{th} fluidic feature for a period of time (e.g., 60 seconds). In some non-limiting embodiments, of monitoring pressure in the i^{th} fluidic feature may include monitoring pressure at one or more of the waste well 112 and the vent well 108. In some embodiments, the process 1900 may include a step 1912 of determining whether any anomalies are present in the monitored pressure. In some non-limiting embodiments, the step 1912 may determine than an anomaly is present if there is a decrease in pressure, which may be indicative of a leak in the i^{th} fluidic feature. If an anomaly is detected, the system 500 may reject the loaded LOC device (or component thereof).

In some non-limiting embodiments, the process 1900 may include a step 1914 of opening the valve in communication with an i^{th} fluidic feature. In some embodiments, the process 1900 may proceed to step 1914 if no anomalies are detected in step 1912. In some embodiments, the process 1900 may include a step 1916 of decreasing pressure in the i^{th} fluidic feature to a negative proof pressure (e.g., −2 psig). In some embodiments, the process 1900 may include a step 1918 of closing the valve. In some embodiments, the process 1900 may include a step 1920 of monitoring pressure in the i^{th} fluidic feature for a period of time (e.g., 60 seconds). In some non-limiting embodiments, of monitoring pressure in the i^{th} fluidic feature may include monitoring pressure at one or more of the waste well 112 and the vent well 108. In some embodiments, the process 1900 may include a step 1922 of determining whether any anomalies are present in the monitored pressure. In some non-limiting embodiments, the step 1922 may determine than an anomaly is present if there is an increase in pressure, which may be indicative of a leak in the i^{th} fluidic feature. If an anomaly is detected, the system 500 may reject the loaded LOC device (or component thereof). In some embodiments, the process 1900 may include a step 1924 in which the system 500 determines whether i=n. In some non-limiting embodiments, n may be equal to the number of fluidic features (e.g., channels) in the LOC device (or component thereof). In some embodiments, the process 1900 may proceed to step 1924 if no anomalies are detected in step 1922. If the system 500 determines that i≠n, the system 500 may increment i and then loop back to step 1904. If the system 500 determines that i=n, the system 500 may determine that the LOC device (or component thereof) has passed the proof test.

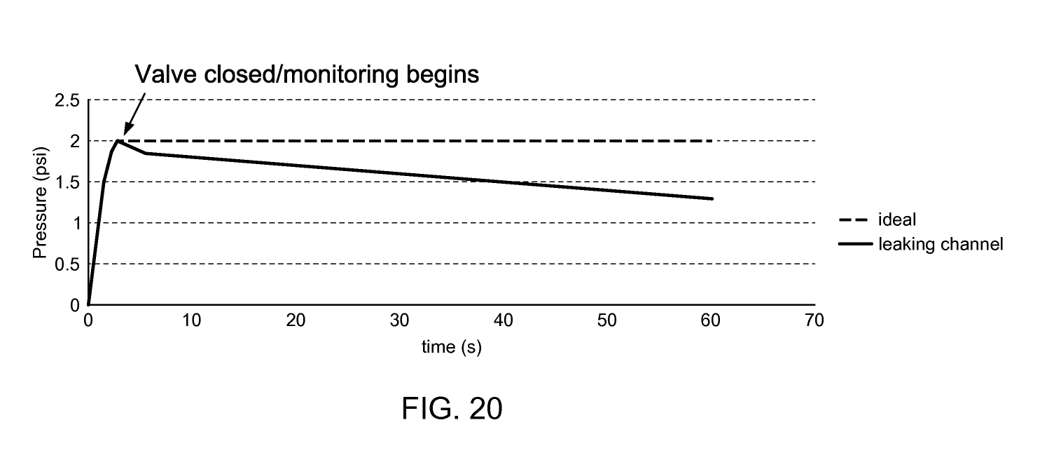

FIG. 20 is a graph illustrating simulated pressure data from a positive proof pressure test (e.g., steps 1904-1912 of FIG. 19). As shown in FIG. 20, in an ideal sealed fluidic feature, the pressure does not change. However, in a leaking fluidic feature, the pressure changes.

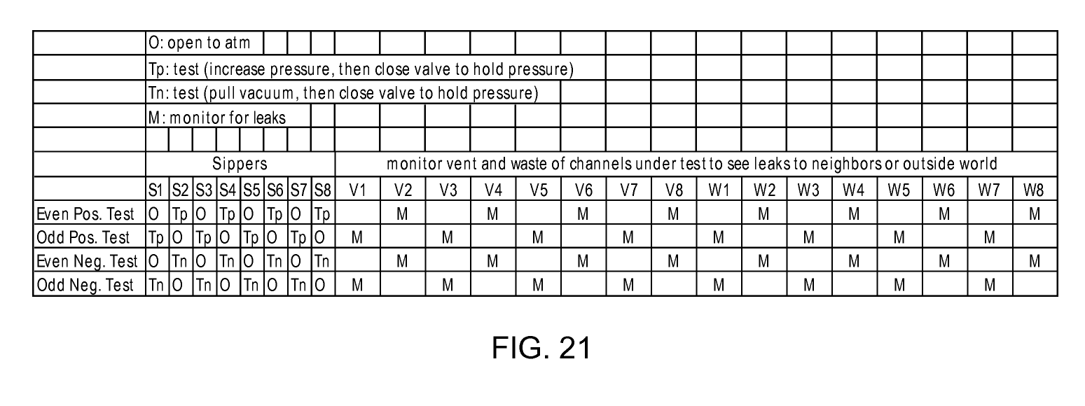

In some embodiments, the system 500 may proof test one or more fluidic features individually, as described with reference to FIG. 19. However, this is not required, and, in some alternative embodiments, the system 500 may test more than one fluidic feature at a time to reduce testing time. For example, in one non-limiting embodiment, as illustrated in FIG. 21, the system 500 may test channels of the fluidic device 101 in two sets with a first set including odd channels and a second set including even channels.

In some embodiments, the LOC test and/or burn-in system 500 may determine premature device failures of LOC devices (so called “infant mortality” failures). In some embodiments, the system 500 may determine fluidic cross-talk (i.e., leaking) between two or more channels (e.g., microchannels) on an LOC device. In some embodiments, the system 500 may determine fluidic cross-talk (i.e., leaking) between two or more blind sample wells or surface reservoirs. In another aspect, the sytem 500 may determine the structural integrity (e.g., sealing from the outside world) of sample wells, buried and surface reservoirs, reaction chambers, channels, and/or microchannel networks. In some non-limiting embodiments, the system 500 may pressure test one or more of these fluidic features at elevated differential pressure (positive and/or negative) to validate the device under more harsh conditions than would normally be experienced during device usage. In some embodiments, the system 500 may enable the testing of one or more components of LOC devices or other microfluidic devices in phases during the production of the complete device assembly. In some embodiments, the system 500 may perform methods for accelerated testing of LOC devices to determine failures that may occur after significant usage. In some embodiments, the system 500 may thermally cycle an LOC device or component thereof to determine failures that may occur during shipment or routine usage. In some embodiments, the system 500 may test electrical circuitry along with fluidic features during the same test and/or burn-in of a device or component. In some embodiments, the system 500 may perform burn-in testing of LOC devices in which device usage is simulated during device test. In some embodiments, the system 500 may be configured to test and/or burn-in complete LOC devices and/or the components used in the assembly of LOC devices.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

What is claimed is:

1. A method of testing a fluidic device comprising fluidic features, the method comprising:
subjecting one or more of the fluidic features to a differential pressure;
measuring a pressure response of one or more of the fluidic features to the differential pressure; and
detecting whether an abnormality is present in the pressure response.

2. The method of claim 1, wherein one or more of the fluidic features comprise a channel.

3. The method of claim 1, wherein one or more of the fluidic features comprise a sample well.

4. The method of claim 1, wherein measuring the pressure response comprises measuring the pressure response of one or more fluidic features that were not subjected to the differential pressure.

5. The method of claim 1, wherein the differential pressure is positive.

6. The method of claim 1, wherein the differential pressure is negative.

7. The method of claim 1, wherein the fluidic device is a sub-component of a lab-on-a-chip device.

8. The method of claim 1, further comprising testing one or more electrical features of the fluidic device, wherein the testing is performed at the same time as or serially with one or more of subjecting the one or more of the fluidic features to the differential pressure, measuring the pressure response, and detecting whether the abnormality is present.

9. The method of claim 8, wherein the one or more electrical features comprise a resistor.

10. The method of claim 1, further comprising subjecting the fluidic device to a thermal profile.

11. The method of claim 10, wherein subjecting the fluidic device to the thermal profile comprises powering one or more features included in or on the fluidic device.

12. The method of claim 10, wherein subjecting the fluidic device to the thermal profile comprises using an environmental chamber or heater that is external to the fluidic device.

13. The method of claim 10, wherein the thermal profile comprises a temperature ramp.

14. The method of claim 10, wherein the thermal profile comprises one or more temperature steps or PCR temperature cycles.

15. The method of claim 1, further comprising subjecting the fluidic device to a humidity and/or pressure profile.

16. The method of claim 1, wherein subjecting the one or more fluidic features to the differential pressure comprises applying the differential pressure to two or more fluidic features at the same time.

17. The method of claim 1, further comprising introducing a liquid into at least one fluidic feature.

18. The method of claim 1, further comprising passing a current through one or more electrical features of the fluidic device.

19. The method of claim 18, wherein the electrical features comprise one or more of a heater, sensor, resistor, capacitor, controller, counter, timer, memory, processor, actuator, and valve.

20. The method of claim 18, wherein passing the current through the one or more electrical features comprises running a burn-in program.

21. The method of claim 20, wherein the burn-in program simulates normal fluidic device usage.

22. The method of claim 20, wherein the burn-in program comprises running the fluidic device at a temperature higher than a standard operating temperature for the device.

23. A method for testing a channel in a fluidic device for leakages, the method comprising:
opening a valve in communication with the channel, wherein the channel is in communication with one or more wells;
subjecting the channel to a proof pressure;
closing the valve; and
monitoring pressure at one or more of the wells, wherein a change in pressure at one or more of the wells is indicative of a leak in the channel.

24. The method of claim 23, wherein the proof pressure is a negative proof pressure, and an increase in pressure at one or more of the wells is indicative of a leak in the channel.

25. The method of claim 23, wherein the proof pressure is a positive proof pressure, and a decrease in pressure at one or more of the wells is indicative of a leak in the channel.

26. The method of claim 23, wherein the channel is in communication with a waste well and a vent well, and monitoring the pressure at one or more of the wells comprises monitoring pressure at one or more of the waste and vent wells.

27. A system for testing a fluidic device comprising fluidic features, the system comprising:
one or more valves or accumulators;
one or more pressure monitors;
a device interface module configured to hold the fluidic device, connect the one or more valves or accumulators to one or more of the fluidic features, and connect the one or more pressure monitors to one or more of the fluidic features; and
a pressure controller configured to control the one or more valves or accumulators to subject one or more of the fluidic features to a differential pressure and control the one or more pressure monitors to measure a pressure response of one or more of the fluidic features.

28. The system of claim 27, wherein one or more of the fluidic features comprise a channel.

29. The system of claim 27, wherein one or more of the fluidic features comprise a sample well.

30. The system of claim 27, wherein the differential pressure is positive.

31. The system of claim 27, wherein the differential pressure is negative.

32. The system of claim 27, wherein the fluidic device is a sub-component of a lab-on-a-chip device, and the device interface module is configured to hold the lab-on-a-chip device.

33. The system of claim 27, further comprising a system controller configured to detect whether an abnormality is present in the pressure response.

34. The system of claim 33, wherein the system controller is configured to control the pressure controller.

35. The system of claim 33, further comprising a storage medium, wherein the system controller is configured store test results in the storage medium.

36. The system of claim 33, further comprising a graphical user interface, wherein the system controller is configured to present test results to a user using the graphical user interface.

37. The system of claim 33, further comprising a circuit tester configured to test one or more electrical features of the fluidic device.

38. The system of claim 37, wherein the system controller is configured to control the circuit tester to test the one or more electrical features at the same time as or serially with subjecting the one or more of the fluidic features to the differential pressure or measuring the pressure response.

39. The system of claim 37, wherein the one or more electrical features comprise a resistor.

40. The system of claim 37, wherein the system controller is configured to control the circuit tester to subject the fluidic device to a thermal profile.

41. The system of claim 40, wherein subjecting the fluidic device to the thermal profile comprises powering one or more features included in or on the fluidic device.

42. The system of claim 40, wherein the thermal profile comprises a temperature ramp.

43. The system of claim 40, wherein the thermal profile comprises one or more temperature steps or PCR temperature cycles.

44. The system of claim 37, wherein the circuit tester is configured to pass a current through one or more electrical features of the fluidic device.

45. The system of claim 44, wherein the electrical features comprise one or more of a heater, sensor, resistor, capacitor, controller, counter, timer, memory, processor, actuator, and valve.

46. The system of claim 37, wherein the system controller is configured to control the electrical tester to burn-in the fluidic device, and the burn-in comprises passing a current through the one or more electrical features of the fluidic device.

47. The system of claim 33, further comprising an environmental chamber or heater that is external to the fluidic device, wherein the system controller is configured to subject the fluidic device to a thermal profile by using the environmental chamber or the external heater.

48. The system of claim 33, further comprising an environmental controller configured to control the environmental conditions under which testing is performed.

49. The system of claim 48, wherein the system controller is configured to control the environmental controller to subject the fluidic device to a humidity and/or pressure profile.

50. The system of claim 27, wherein subjecting the one or more fluidic features to the differential pressure comprises applying the differential pressure to two or more fluidic features at the same time.

51. The system of claim 27, wherein the pressure response is of one or more fluidic features that were not subjected to the differential pressure.

52. A system for testing a channel in a fluidic device for leakages, the system comprising:
a valve;
one or more pressure monitors;
a device interface module configured to hold the fluidic device, connect the valve to the channel of the fluidic device, and connect the one or more pressure monitors to one or more wells in communication with the channel;
a pressure controller configured to open and close the valve, to subject the channel to a proof pressure, and to control the one or more pressure monitors to measure a pressure at one or more of the wells; and
a system controller configured to (i) control the pressure controller to open the valve, subject to the channel to the proof pressure, close the valve, and control the one or more pressure monitors to measure a pressure at one or more of the wells, and (ii) determine whether the measured pressure at one or more of the wells changes, wherein a change in pressure at one or more of the wells is indicative of a leak in the channel.
